(19) United States
(12) Patent Application Publication
(10) Pub. No.: US 2010/0143322 A1
(43) Pub. Date: Jun. 10, 2010

(54) USE OF INHIBITORS OF N-METHYL TRANSFERASES FOR THE THERAPY OF PARKINSON'S DISEASE

(76) Inventors: Hans Uwe Wolf, Neu-Ulm (DE); Christoph Wilhelm, Ulm (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/993,738

(22) PCT Filed: Jun. 26, 2006
(86) PCT No.: PCT/EP06/06132

§ 371 Date: Feb. 16, 2010

(30) Foreign Application Priority Data
Jun. 24, 2005 (DE) 10 2005 029 468.5

(51) Int. Cl. A61K 38/45; A61K 38/02; A61K 38/06; A61K 31/47; A61P 25/16; A61K 31/135; A61K 31/13; A61K 31/55; A61K 31/5513; A61K 31/553; A61K 31/554

(52) U.S. Cl. 424/94.5; 514/2; 514/18; 514/307; 514/310; 514/309; 514/646; 514/659; 514/213.01; 514/221; 514/211.09

(57) ABSTRACT

The present invention relates to the use of inhibitors of different N-methyl transferases in the therapy of Parkinson's syndrome, in particular idiopathic Parkinson's syndrome.

The subject of the present invention is the use of inhibitors of different N-methyl transferases in the therapy of Parkinson's syndrome, in particular idiopathic Parkinson's syndrome.

Parkinson's syndrome (PS) is after Alzheimer's disease the most widespread neurodegenerative disease which is manifested in humans on average from the age of 57. The probability of suffering from a PS increases with increasing age and, for 65 year olds, is 1-2%. The number of PS patients in most countries is between 0.5-2%, approximately 15,000 new patients being added annually. PS is not sex-specific.

The pathogenesis of PS is still extensively unexplained in detail despite extensive scientific works. However it is certain that, in the course of the pathogenesis, specific brain regions, in particular the substantia nigra, are damaged. The melanin-containing neurons in this region are destroyed and, on the other hand, the concentration of the neurotransmitter dopamine is lowered in general.

Since approx. 1983, there have been clear references to the initiation by or the joint involvement of neurotoxins in the pathogenesis of PS. The starting point of the suspicions occurring at that time was the observation that an exogenous substance can obviously initiate a PS. Drug addicts who had taken a home-made heroin replacement substance developed symptoms of PS within a few days. Methyl-4-phenyl-1,2,3,6-tetrahydropyridine (MPTP), an impurity of the mentioned heroin replacement substance, was found to be the cause of these symptoms (Langston, 1983).

MPTP has a methyl group on an N-ring atom. The uncharged molecule MPTP can cross the blood-brain barrier and pass into the astrocytes. It is converted there enzymatically to form a cation. By means of the dopamine transport system this cation passes into the dopaminergic neurons where it is bonded to neuromelanin. By means of a further transport system it passes into the mitochondria. There it interrupts the respiratory chain. Consequently, the result is destruction of the cell.

After MPTP in the form of the cation formed therefrom had been established as a PS-initiating agent, the suspicion was obvious that also MPTP-similar substances could initiate a PS, irrespective of whether they are of an exogenous or endogenous origin. This would mean that neurotoxins have a possible general relevance in the pathogenesis of PS.

Isoquinolines and β-carbolines are endogenous MPTP-similar substances which have been found in post-mortem brains and cerebrospinal fluids of PS patients. Even in 1970 it was suspected that tetrahydroisoquinolines (“THIQ”) are formed in the brain of mammals. It was assumed that exogenously administered DOPA (3,4-dihydroxyphenylalanine) is converted into dopamine which is oxidised by monoamine oxidase into 3,4-dihydroxyphenylacetaldehyde. This reacts with dopamine in a so-called Pictet-Spengler reaction to form tetrahydropapaveroline.

Since the discovery of MPTP as a potent PS-initiating neurotoxin, the existence of structural similarities between N-methyltetrahydroisoquinolines (“N-Me-THIQ”) and N-methyltetrahydro-β-carbolines (“N-Me-β-THBC”) led to the hypothesis that these compounds form a group of neurotoxins which can lead to PS (Collins and Neafsay, 1985; Nagatsu and Yoshida, 1988; Nagatsu et al., 1994). N-Me-THIQ was found post-mortem in the brains of Parkinson's patients.

The mentioned compound groups of N-Me-THIQ and N-Me-β-THBC were present after absorption into the organism or after synthesis thereof in the organism, firstly in a non-methylated form. This form shows, if at all, a clearly lower toxicity than the neurotoxins themselves. They can therefore be termed to be pre-neurotoxins.

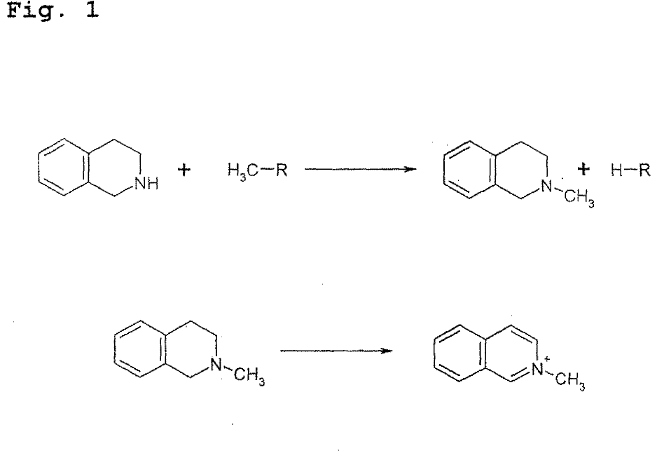

The mentioned pre-neurotoxins are converted in the human organism by enzymatically controlled reactions into the active neurotoxins (ultimate neurotoxins). This conversion is effected in two toxicating partial steps. Firstly, methylation is effected on the ring nitrogen atom. In a second reaction, the tetrahydroisoquinoline- or the tetrahydro-β-carboline basic framework is oxidised, which leads to formation of the isoquinoline- and β-carboline basic frameworks with respectively one quaternary, positively charged nitrogen atom. This structure is, analogously to the MPTP cation (see above), the actually effective form of the neurotoxin. The reaction mechanism is represented in FIG. 1.

The described methylation reaction hence represents a crucial partial reaction of the toxication.

The methylisation reaction is effected enzymatically with involvement of specific N-methyl transferases (NMT), in particular phenylethanolamine N-methyl transferase (PNMT) (EC 2.1.1.28), in addition nicotinamide N-methyl transferase (EC 2.1.1.1), nicotinate N-methyl transferase (EC 2.1.1.7), histamine N-methyl transferase (EC 2.1.1.8), glycine N-methyl transferase (EC 2.1.1.20), tyramine N-methyl transferase (EC 2.1.1.27), dimethylhistidine N-methyl transferase (EC 2.1.1.44), amine N-methyl transferase (EC 2.1.1.49), dimethylhistidine N-methyl transferase (EC 2.1.1.44), calmodulin-lysine N-methyl transferase (EC 2.1.1.60), (8)-tetrahydroproto-berberine N-methyl transferase (EC 2.1.1.122) and histone-arginine N-methyl transferase (EC 2.1.1.125). These enzymes are present in cells of numerous organs of the human organism.

Starting herefrom, it was the object of the present invention to provide a drug with which formation of neurotoxins in Parkinson's syndrome is prevented.

This object is achieved by the features of claim 1. The further dependent claims reveal advantageous developments.

According to the invention, at least one exogenous or endogenous inhibitor of N-methyl transferases is used to produce a drug for the therapy and the prophylaxis of Parkinson's syndrome.

Within the scope of the pathogenesis of Parkinson's syndrome, significant importance is attributed to the inhibitors according to the invention. An inhibitor of this type has the capacity to inhibit the above-described first partial step of the toxic reaction of the pre-neurotoxin to the neurotoxin and hence to reduce or to prevent the formation of the ultimate neurotoxins. Hence such an inhibitor can eliminate one of the causes of the development of PS.

The bonding of these inhibitors to the receptor which is suitable for this purpose on the NMT leads to effective inhibition of these enzymes which is responsible for the first partial step of the toxication of the pre-neurotoxins into the actually effective ultimate neurotoxins. Since the PS develops as a consequence of an accumulation of damage due to the neurotoxins over a period of years, possibly decades, the stationary concentration of resulting neurotoxins is permanently lowered by the effect of the mentioned inhibitors and consequently the progress of PS is slowed down or stopped. A cure for PS is possible in principle if the nerve cells which are missing as a result of the process of cell damage or cell destruction which has been proceeding up till then can be replaced again by new formation of cells.

A preferred variant of the teaching according to the invention provides that the at least one inhibitor inhibits the enzymatic activity of N-methyl transferases, which is selected from the group comprising phenylethanolamine N-methyl transferase (PNMT) (EC 2.1.1.28), nicotinamide N-methyl transferase (EC 2.1.1.1), nicotinate N-methyl transferase (EC 2.1.1.7), histamine N-methyl transferase (EC 2.1.1.8), glycine N-methyl transferase (EC 2.1.1.20), tyramine N-methyl transferase (EC 2.1.1.27), dimethylhistidine N-methyl transferase (EC 2.1.1.44), amine N-methyl transferase (EC 2.1.1.49), dimethylhistidine N-methyl transferase (EC 2.1.1.44), calmodulin-lysine N-methyl transferase (EC 2.1.1.60), (S)-tetrahydroproto-berberine N-methyl transferase (EC 2.1.1.122), and histone-arginine N-methyl transferase (EC 2.1.1.125).

The inhibitors according to the invention belong in particular to the group of oligopeptides, 1,2,3,4-tetrahydroisoquinolines (1,2,3,4-THIQ), phenylethanolamines, tetrahydro-1H-2-benzazepines, tetrahydro-5H-1,4-benzoxazepines, phenylethanolamines and cycloalkylethylamines.

In the case of the oligopeptides, compounds are concerned which are constructed from two to 30 amino acids in peptidic cross-linking (so-called peptide structure). The relevant compounds display high affinity to a receptor on the surface of various N-methyl transferases, in particular PNMT.

Examples of inhibitors from the group of oligopeptides is on the one hand the tripeptide Ala-Cys-Cys which has proved itself to be a very effective inhibitor of PNMT. Thus even by means of less than 2 mg of the Ala-Cys-Cys, isolated from rabbit liver and purified, more than a 90% inhibition of the PNMT occurs in the PNMT test (Chr. Wilhelm, Dissertation Dr. biol. hum., University of Ulm, 2005).

A further example is the inhibitor isolated from rat liver by Hong et al. (1986) which comprises 27 amino acids, Hong S Y, Lee H W, Desi S, Kim S, Paik W K (1986): Eur J Biochem 156: 79-84). In contrast to the inhibitor isolated by Wilhelm, the inhibitor described by Hong contains a fluorescent chromophore.

For therapeutic use, the described tripeptide is however more suitable than the comparatively large inhibitor described by Hong et al., since difficulties during synthesis and problems with respect to a possible sensitivisation with increasing molecular size increase noticeably.

As examples of NMT inhibitors from the group of 1,2,3,4-THIQ, the basic substance 1,2,3,4-THIQ and numerous derivatives should be mentioned, thus for example (R)-3-methyl-1,2,3,4-THIQ, (S)-3-methyl-1,2,3,4-THIQ (hydrochloride), 3-trifluoromethyl-1,2,3,4-THIQ, 3-fluoromethyl-1,2,3,4-THIQ, 3-trifluoromethyl-7-bromo-1,2,3,4-THIQ, 3-trifluoromethyl-7-cyano-1,2,3,4-THIQ, 3-trifluoromethyl-7-nitro-1,2,3,4-THIQ, 3-fluoromethyl-7-(N-benzylamino-sulphonyl)-1,2,3,4-THIQ, 3-fluoromethyl-7-(N-methyl-aminosulphonyl)-1,2,3,4-THIQ, 3-fluoromethyl-7-[N-(4-chlorophenyl)aminosulphonyl]-1,2,3,4-THIQ, 3-fluoromethyl-7-aminosulphonyl)-1,2,3,4-THIQ, 3-fluoromethyl-7-azido-1,2,3,4-THIQ, 3-fluoromethyl-7-bromo-1,2,3,4-THIQ, 3-fluoromethyl-7-cyano-1,2,3,4-THIQ, 3-fluoromethyl-7-iodo-1,2,3,4-THIQ, 3-fluoromethyl-7-isothio-cyanato-1,2,3,4-THIQ, 3-fluoromethyl-7-methanesulphonyl-1,2,3,4-THIQ, 3-fluoromethyl-7-nitro-1,2,3,4-THIQ, 3-fluoromethyl-7-trifluoromethyl-1,2,3,4-THIQ, 1,2,3,4-THIQ-7-carboxylic acid (CAS 41034-52-0), 7-acetamido-1,2,3,4-THIQ, 7-allylsulphonyl-1,2,3,4-THIQ, 7-aminocarbonyl-1,2,3,4-THIQ, 7-aminomethyl-1,2,3,4-THIQ (dihydrochloride), 7-benzoyl-1,2,3,4-THIQ, 7-benzyl-1,2,3,4-THIQ, 7-bromo-N-triphenylmethyl-1,2,3,4-THIQ, 7-hydroxymethyl-1,2,3,4-THIQ oxalate, 7-iodo-1,2,3,4-THIQ, 7-methoxycarbonyl-1,2,3,4-THIQ, 7-methylsulphinyl-1,2,3,4-THIQ, 7-methylsulphonyl-1,2,3,4-THIQ, 7-methylthio-1,2,3,4-THIQ, 7-phenylsulphonyl-1,2,3,4-THIQ, 7-trichloromethylsulphonyl-1,2,3,4-THIQ, 7-trifluoroacetyl-1,2,3,4-THIQ (hydrochloride), 7-methylsulphonyl-3-trifluoromethyl-1,2,3,4-THIQ, 7,8-dichloro-1,2,3,4-THIQ (SKF-64139), 3-chloromethyl-1,2,3,4-THIQ and 3-hydroxymethyl-1,2,3,4-THIQ.

As examples of NMT inhibitors, from the group of phenylethanolamines there should be mentioned 2-chlorophenylethanolamine, 3,4 dichlorophenylethanolamine, 2-fluororophenylethanolamine, 3,4 dihydroxyphenylethanolamine, 3-bromophenylethanolamine, 4-bromophenylethanolamine, 4-fluorophenylethanolamine and 4-hydroxyphenylethanolamine.

Examples of inhibitors from the group of tetrahydrobenzazepines, of -benzodiazepines and benzoxazepines are: 2,3,4,5-tetrahydro-1H-2-benzazepine (CAS 1701-57-1), 3-alkyl-tetrahydro-1H-2-benzazepine, 4-hydroxy-tetrahydro-1H-2-benzazepine, 8-aryl-4-fluoro-tetrahydro-1H-2-benzazepine, 8,9-dichloro-2,3,4,5-tetrahydro-1H-2-benzazepine (LY134046) (CAS 71274-97-0), 3-methyl-8,9-dichloro-2,3,4,5-tetrahydro-1H-2-benzazepine and 8-substituted derivatives of 4-fluoro-2,3,4,5-tetrahydro-1H-2-benzazepine, in addition 2,3,4,5-tetrahydro-5H-1,4-benzodiazepine, 2,3,4,5-tetrahydro-5H-1-4-benzothiazepine and 2,3,4,5-tetrahydro-5H-1-4-benzoxazepine.

As examples of compounds from the group of cycloalkylethylamines, there should be mentioned 2-cyclooctyl-2-ethylamine and 2-cyclohexyl-2-hydroxyethylamine.

Further inhibitors of various NMT which are suitable in principle for the therapy of PS are: 1-aminomethylcycloundecanol, 2-(aminomethyl)-trans-2-decalol, 2,3-dichloro-α-methylbenzylamine, metoprine, 4-(N,N)-dimethylamino)butylisothio-urea (SKF 91488), 3,4-dichlorophenylethylenediamine, 2,5-dimethyl-1-aminobenzamidazole, octopamine (CAS 104-14-3, CAS 876-04-0), sinefungin (CAS 58944-73-3), 2-aminotetralin (CAS 2954-50-9), 5,6-dichloro-2-aminotetralin, 3,4-dichloroamphetamine, berberine, N,N-dimethyltryptamine, calmidazolium, imidazolepropionate, 5-methyltetrahydrofolate hexaglutamate, 5-methyltetrahydrofolate pen taglutamate, 5-methyltetrahydrofolate triglutamate, 5-methyltetrahydrofolic acid, folinic acid, 5′-[p-(fluorosulphonyl)benzoyl]adenosine, 5,5′-dithiobis-(2-nitrobenzoate), 5-methyltetrahydropteroylpentaglutamate, bromolysergic acid-diethylamide, bufotenin, tubocurare, amodiaquine, d-chlorpheniramine, dimaprit, impromidine, 3-deazaadenosine, 5′-methyl-thioadenosine A9145C, n-butyl-thioadenosine, S-inosyl-L-(2-hydroxy-4-methylthio)-butyrate, thioethanoladenosine, N1-methylnicotinamide, picolinic acid, pyrazinamide, trigonelline, homotyramine and N-methyltyramine.

With reference to the subsequent example and the subsequent Figures, the subject according to the invention is intended to be explained in more detail, without wishing to restrict the latter to the particular embodiments shown here.

### EXAMPLE

### Purification and Characterisation of an Inhibitor of Phenylethanolamine N-Methyl Transferase from Rabbit Liver

The above-indicated inhibitor comprising 1 mol alanine and 2 mol cysteine was purified, isolated and characterised in the following manner:

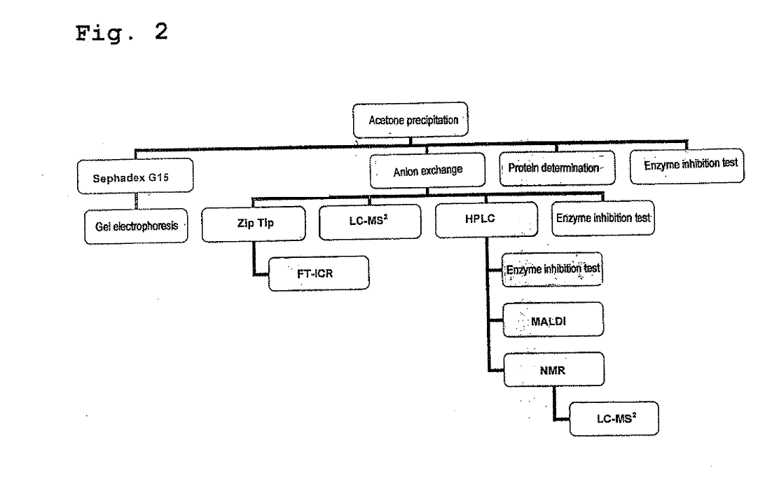

The rabbit liver was firstly homogenised in 10 mmol/l tris and 0.1 mmol/l EDTA (pH 7.3). The subsequent purification and isolation was effected firstly by twice-repeated centrifugation in which coarse cell components were removed. Following thereon, there was an acetone precipitation for removing the dissolved inert protein. For further purification steps and for concentration, anion exchange and HPLC were used. In all cleaning steps, an enzymatic activity determination of the inhibitor was effected. Determination of the relative molar mass of the inhibitor was effected by LC-MS2 (liquid chromatography with twofold mass spectrometry coupling) and FT-ICR (Fourier transform ion cyclotron resonance). FIG. 2 gives an overview of the purification and characterisation steps which were implemented.

Detection of the inhibitor was effected by inhibiting the enzyme PNMT (phenylethanolamine N-methyl transferase). Using the radioactively marked methyl donor S-adenosyl methionine (SAM), normetanephrine was converted into metanephrine by means of PNMT. In the enzyme inhibiting test, the radioactivity of the H3-marked methyl group, which stems from SAM, in metanephrine was measured.

In at least one fraction of all the purification steps, a significant inhibition of the PNMT occurred, which permitted simple monitoring of the purification process. The residual activity of the enzyme was thereby lowered to values of ≧10% of the enzyme control value.

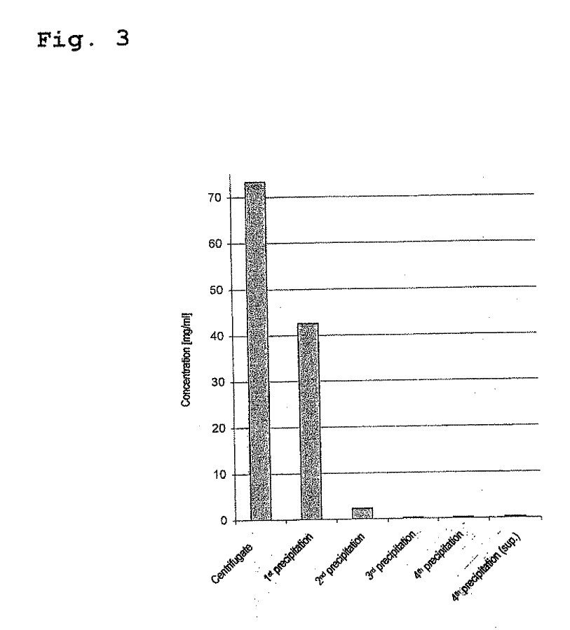

Determination of the proportion of foreign protein in the individual fractions was effected by a protein determination. The protein concentration of the individual fractions from the acetone precipitation is represented in FIG. 3. The protein concentrations are represented here for the precipitations of the various acetone precipitations and of the supernatant (sup) of the 4th acetone precipitation. The protein concentration reduces significantly, as anticipated, in the course of the acetone fractionation, for example from 73.1 μg/ml in the centrifugate before the first precipitation to 0.22 μg/ml in the supernatant of the 4^{th} precipitation. The resulting purification factor of the supernatant of the 4^{th} precipitation relative to the centrifugate is therefore approx. 300.

Further purification steps were effected with the help of an anion exchanger in two steps at pH 7.0 and pH 10.0 with a KCl— or an NaCl gradient of 0 to 3 mol/l and also by high pressure liquid chromatography at pH 6.0 on a Reprosil-Pur C18-AQ column with the help of a methanol gradient.

Determination of the relative molar mass was effected with the help of the LC-MS2 (liquid chromatography with two-stage mass spectrometry coupling) and the FT-ICR (Fourier transform ion cyclotron resonance). The result was a value of 295 in accordance with the composition of the inhibitor of 1 mol alanine and 2 mol cysteine.

1. A method for the treatment or prophylaxis treating of Parkinson's syndrome comprising inhibiting N-methyl transferases using at least of one exogenic or endogenic inhibitor of N-methyl transferases to prevent the formation of Parkinson's syndrome neurotoxins via methylation of non-methylated pre-neurotoxins.

2. A method according to claim 1, wherein said at least one exogenic or endogenic inhibitor is selected from phenylethanolamine N-methyl transferase (PNMT) (EC 2.1.1.28), nicotinamide N-methyl transferase (EC 2.1.1.1), nicotinate N-methyl transferase (EC 2.1.1.7), histamine N-methyl transferase (EC 2.1.1.8), glycine N-methyl transferase (EC 2.1.1.20), tyramine N-methyl transferase (EC 2.1.1.27), dimethylhistidine N-methyl transferase (EC 2.1.1.44), amine N-methyl transferase (EC 2.1.1.49), dimethylhistidine N-methyl transferase (EC 2.1.1.44), calmodulin-lysine N-methyl transferase (EC 2.1.1.60), (S)-tetrahydroproto-berberine N-methyl transferase (EC 2.1.1.122), and histone-arginine N-methyl transferase (EC 2.1.1.125).

3. A method according to claim 1, wherein said at least one exogenic or endogenic inhibitor is an oligopeptide, a oligopeptide derivative, or a mixture thereof.

4. A method according to claim 1, wherein said at least one exogenic or endogenic inhibitor is a tripeptide, a tripeptide derivative, or a mixture thereof.

5. A method according to claim 4, wherein said the at least one exogenic or endogenic inhibitor is selected from alanine- and cysteine-containing tripeptides.

6. A method according to claim 1, wherein said at least one exogenic or endogenic inhibitor is 1,2,3,4-tetrahydroisoquinoline or a derivative thereof.

7. A method according to claim 1, wherein said at least one exogenic or endogenic inhibitor is selected from 1,2,3,4-THIQ, (R)-3-methyl-1,2,3,4-THIQ, (S)-3-methyl-1,2,3,4-THIQ (hydrochloride), 3-trifluoromethyl-1,2,3,4-THIQ, 3-fluoromethyl-1,2,3,4-THIQ, 3-trifluoromethyl-7-bromo-1,2,3,4-THIQ, 3-trifluoromethyl-7-cyano-1,2,3,4-THIQ, 3-trifluoromethyl-7-nitro-1,2,3,4-THIQ, 3-fluoromethyl-7-(N-benzylamino-sulphonyl)-1,2,3,4-THIQ, 3-fluoromethyl-7-(N-methyl-aminosulphonyl)-1,2,3,4-THIQ, 3-fluoromethyl-7-[N-(4-chlorophenyl)aminosulphonyl]-1,2,3,4-THIQ, 3-fluoromethyl-7-aminosulphonyl)-1,2,3,4-THIQ, 3-fluoromethyl-7-azido-1,2,3,4-THIQ, 3-fluoromethyl-7-bromo-1,2,3,4-THIQ, 3-fluoromethyl-7-cyano-1,2,3,4-THIQ, 3-fluoromethyl-7-iodo-1,2,3,4-THIQ, 3-fluoromethyl-7-isothio-cyanato-1,2,3,4-THIQ, 3-fluoromethyl-7-methanesulphonyl-1,2,3,4-THIQ, 3-fluoromethyl-7-nitro-1,2,3,4-THIQ, 3-fluoromethyl-7-trifluoromethyl-1,2,3,4-THIQ, 1,2,3,4-THIQ-7-carboxylic acid (CAS 41034-52-0), 7-acetamido-1,2,3,4-THIQ, 7-allylsulphonyl-1,2,3,4-THIQ, 7-aminocarbonyl-1,2,3,4-THIQ, 7-aminomethyl-1,2,3,4-THIQ (dihydrochloride), 7-benzoyl-1,2,3,4-THIQ, 7-benzyl-1,2,3,4-THIQ, 7-bromo-N-triphenylmethyl-1,2,3,4-THIQ, 7-hydroxymethyl-1,2,3,4-THIQ oxalate, 7-iodo-1,2,3,4-THIQ, 7-methoxycarbonyl-1,2,3,4-THIQ, 7-methylsulphinyl-1,2,3,4-THIQ, 7-methylsulphonyl-1,2,3,4-THIQ, 7-methylthio-1,2,3,4-THIQ, 7-phenylsulphonyl-1,2,3,4-THIQ, 7-trichloromethylsulphonyl-1,2,3,4-THIQ, 7-trifluoroacetyl-1,2,3,4-THIQ (hydrochloride), 7-methylsulphonyl-3-trifluoromethyl-1,2,3,4-THIQ, 7,8-dichloro-1,2,3,4-THIQ (SKF-64139), 3-chloromethyl-1,2,3,4-THIQ and 3-hydroxymethyl-1,2,3,4-THIQ, wherein THIO is tetrahydroisoquinoline.

8. A method according to claim 1, wherein said at least one exogenic or endogenic inhibitor is a phenylethanolamine.

9. A method according to claim 8, wherein said at least one exogenic or endogenic inhibitor is selected from 2 chlorophenylethanolamine, 3,4 dichlorophenylethanolamine, 2-fluororophenylethanolamine, 3,4 dihydroxyphenylethanolamine, 3-bromophenylethanolamine, 4-bromophenylethanolamine, 4-fluorophenylethanolamine, and 4-hydroxyphenylethanolamine.

10. A method according to claim 1, wherein said at least one inhibitor exogenic or endogenic is a tetrahydrobenzazepine, a tetrahydrobenzodiazepine, and/or a tetrahydrobenzoxazepine.

11. A method according to claim 10, wherein at least one exogenic or endogenic inhibitor is selected from 2,3,4,5-tetrahydro-1H-2-benzazepine (CAS 1701-57-1), 3-alkyl-tetrahydro-1H-2-benzazepine, 4-hydroxy-tetrahydro-1H-2-benzazepine, 8-aryl-4-fluoro-tetrahydro-1H-2-benzazepine, 8,9-dichloro-2,3,4,5-tetrahydro-1H-2-benzazepine (LY134046) (CAS 71274-97-0), 3-methyl-8,9-dichloro-2,3,4,5-tetrahydro-1H-2-benzazepine and 8-substituted derivatives of 4-fluoro-2,3,4,5-tetrahydro-1H-2-benzazepine, in addition 2,3,4,5-tetrahydro-5H-1,4-benzodiazepine, 2,3,4,5-tetrahydro-5H-1-4-benzothiazepine, and 2,3,4,5-tetrahydro-5H-1-4-benzoxazepine.

12. A method according to claim 1, wherein said at least one exogenic or endogenic inhibitor is a cycloalkylethylamine.

13. A method according to claim 12, wherein said at least one exogenic or endogenic inhibitor is selected from 2 cyclooctyl-2-ethylamine and 2-cyclohexyl-2-hydroxyethylamine.

14. A method according to claim 1, wherein said at least one exogenic or endogenic inhibitor is selected from 1 aminomethylcycloundecanol, 2-(aminomethyl)-trans-2-decalol, 2,3-dichloro-α-methylbenzylamine, metoprine, 4-(N,N)-dimethylamino)butylisothio-urea (SKF 91488), 3,4-dichlorophenylethylenediamine, 2,5-dimethyl-1-aminobenzamidazole, octopamine (CAS 104-14-3, CAS 876-04-0), sinefungin (CAS 58944-73-3), 2-aminotetralin (CAS 2954-50-9), 5,6-dichloro-2-aminotetralin, 3,4-dichloroamphetamine, berberine, N,N-dimethyltryptamine, calmidazolium, imidazolepropionate, 5-methyltetrahydrofolate hexaglutamate, 5-methyltetrahydrofolate pentaglutamate, 5-methyltetrahydrofolate triglutamate, 5-methyltetrahydrofolic acid, folinic acid, 5′-[p-(fluorosulphonyl)benzoyl]adenosine, 5,5′-dithiobis-(2-nitrobenzoate), 5-methyltetrahydropteroylpentaglutamate, bromolysergic acid-diethylamide, bufotenin, tubocurare, amodiaquine, d-chlorpheniramine, dimaprit, impromidine, 3-deazaadenosine, 5′-methyl-thioadenosine, A9145C, n-butyl-thioadenosine, S-inosyl-L-(2-hydroxy-4-methylthio)-butyrate, thioethanoladenosine, N1-methylnicotinamide, picolinic acid, pyrazinamide, trigonelline, homotyramine, and N-methyltyramine.
